(19) United States
(12) Patent Application Publication
(10) Pub. No.: US 2017/0144670 A1
(43) Pub. Date: May 25, 2017

(54) VEHICLE SAFETY METHOD AND VEHICLE SAFETY SYSTEM

(71) Applicants: Inventec (Pudong) Technology Corporation, Shanghai (CN); INVENTEC CORPORATION, TAIPEI CITY (TW)

(72) Inventors: Hong-You CHOU, TAIPEI CITY (TW); Tai-Yi CHIANG, TAIPEI CITY (TW); Po-Tsun YANG, TAIPEI CITY (TW); Jeng-Ting PAN, TAIPEI CITY (TW)

(21) Appl. No.: 15/156,345

(22) Filed: May 17, 2016

(30) Foreign Application Priority Data
Nov. 24, 2015 (CN) 201510822132.7

(51) Int. Cl. B60W 40/08; A61B 5/00; A61B 5/18

(52) U.S. Cl. CPC B60W 40/08; A61B 5/18; A61B 5/746; A61B 5/741; A61B 5/681; A61B 5/0077; B60W 2040/0818; B60W 2540/26; B60W 2540/04; B60W 2710/18; B60W 2720/24; B60W 2710/30

(57) ABSTRACT

A vehicle safety method includes steps: monitoring a face state of a driver and determining whether the face state satisfies a tired state or not; sensing a physiological state of the driver and determining whether the physiological state satisfies a conscious state or not; sending a warning query when the face state satisfies the fired state and the physiological state satisfies the conscious state; determining whether a response corresponding to the warning query is received or not; and activating a vehicle safety mechanism if the response corresponding to the warning query is not received.

### RELATED APPLICATIONS

This application claims priority to Chinese Application Serial Number 201510822132.7, filed Nov. 24, 2015, which is herein incorporated by reference.

### BACKGROUND

Technical Field

The present disclosure relates to a safety method and a safety system. More particularly, the present disclosure relates to a vehicle safety method and a vehicle safety system.

Description of Related Art

With the development of technology, cars have been a very important role in our daily life. For avoiding an accident due to a driver who is tired, an alarm system has been installed in a car in the present day. The alarm system can send out an alarm signal to remind the driver. For instance, a driver (operator) sensor is installed in a car. The behavior state, pulse state, heartbeat, blood pressure, ethanol concentration of the driver is sensed through the driver sensor based on a legal standard value. However, an accident still occurs when the driver can't receive the alarm signal or the driver can't response immediately.

A prior art is to determine a conscious state of a driver based on heartbeat (pulse state), and to notify a hospital if the driver is determined being unconscious. However, if a sensor is damaged and is unable to determine whether the driver really loses heartbeat or whether the driver is really unconscious or not, it doesn't need to notify the hospital immediately if the driver is still conscious.

### SUMMARY

One embodiment of the present disclosure is related to a vehicle safety method. The vehicle safety method includes steps: monitoring a face state of a driver and determining whether the face state satisfies a tired state or not; sensing a physiological state of the driver and determining whether the physiological state satisfies a conscious state or not; sending a warning query when the face state satisfies the tired state and the physiological state satisfies the conscious state; determining whether a response corresponding to the warning query is received or not; activating a vehicle safety mechanism if the response corresponding to the warning query is not received.

One embodiment of the present disclosure is related to a vehicle safety system. The vehicle safety system includes a face monitoring device, a physiology sensing device, and a vehicle computer. The face monitoring device is configured to monitor a face state of a driver. The physiology sensing device is electrically connected to the face monitoring device to sense a physiological state of the driver. The vehicle computer electrically connected to the face monitoring device and the physiology sensing device. The vehicle computer is configured to determine whether the face state satisfies a tired state or not and determine whether the physiological state satisfies a conscious state or not. The vehicle computer sends a warning query and determines whether a response corresponding to the warning query is received or not if the face state satisfies the tired state and the physiological state satisfies the conscious state. The vehicle computer activates a vehicle safety mechanism if the response corresponding to the warning query is not received.

As the above embodiments, the vehicle safety method and the vehicle safety system send warning query when the driver is tired and conscious and activate the vehicle safety mechanism if the response corresponding to the warning query is not received. Consequently, the vehicle safety mechanism is configured to avoid an occurrence of an accident if the driver doesn't send the response (for example, the driver is sleeping).

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows.

FIG. 1 is a flow diagram illustrating a vehicle safety method according to one embodiment of the present disclosure;

FIG. 2 is a schematic diagram illustrating a vehicle safety system according to one embodiment of this disclosure;

FIG. 3 is a schematic diagram illustrating a vehicle safety system according to another embodiment of this disclosure;

FIG. 4 is a flow diagram illustrating a vehicle safety method according to yet another embodiment of this disclosure; and

FIG. 5 is a schematic diagram illustrating a vehicle safety system according to yet another embodiment of this disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts. The embodiments below are described in detail with the accompanying drawings, but the examples provided are not intended to limit the scope of the disclosure covered by the description. The structure and operation are not intended to limit the execution order. Any structure regrouped by elements, which has an equal effect, is covered by the scope of the present disclosure.

In addition, the drawings are for the purpose of illustration only, and are not in accordance with the size of the original drawing. The components in description are described with the same number to understand.

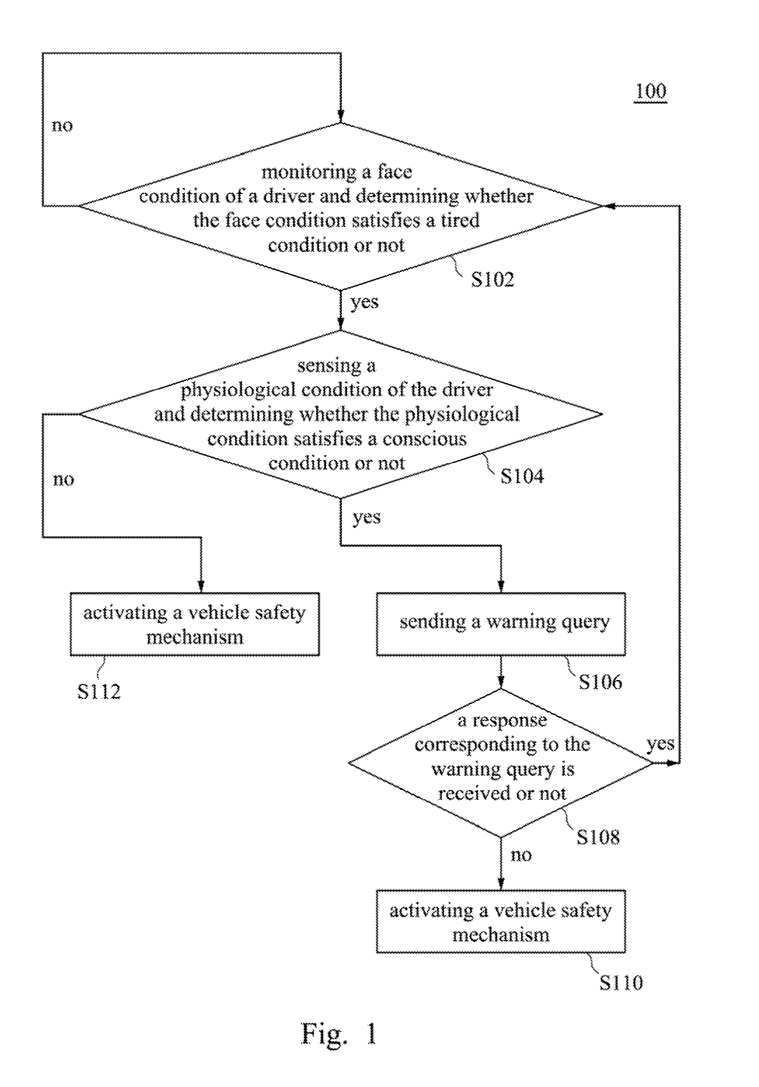

FIG. 1 is a flow diagram illustrating a vehicle safety method 100 according to one embodiment of the present disclosure. The vehicle safety method 100 is configured to send a warning query when a driver is tired but is conscious. A vehicle safety mechanism is automatically activated if the driver doesn't response the warning query. Thus, the vehicle safety mechanism may be configured to protect the driver. As illustrated in FIG. 1, the vehicle safety method 100 includes at least steps S102-S110. In some embodiments, the vehicle safety method 100 further includes step S112.

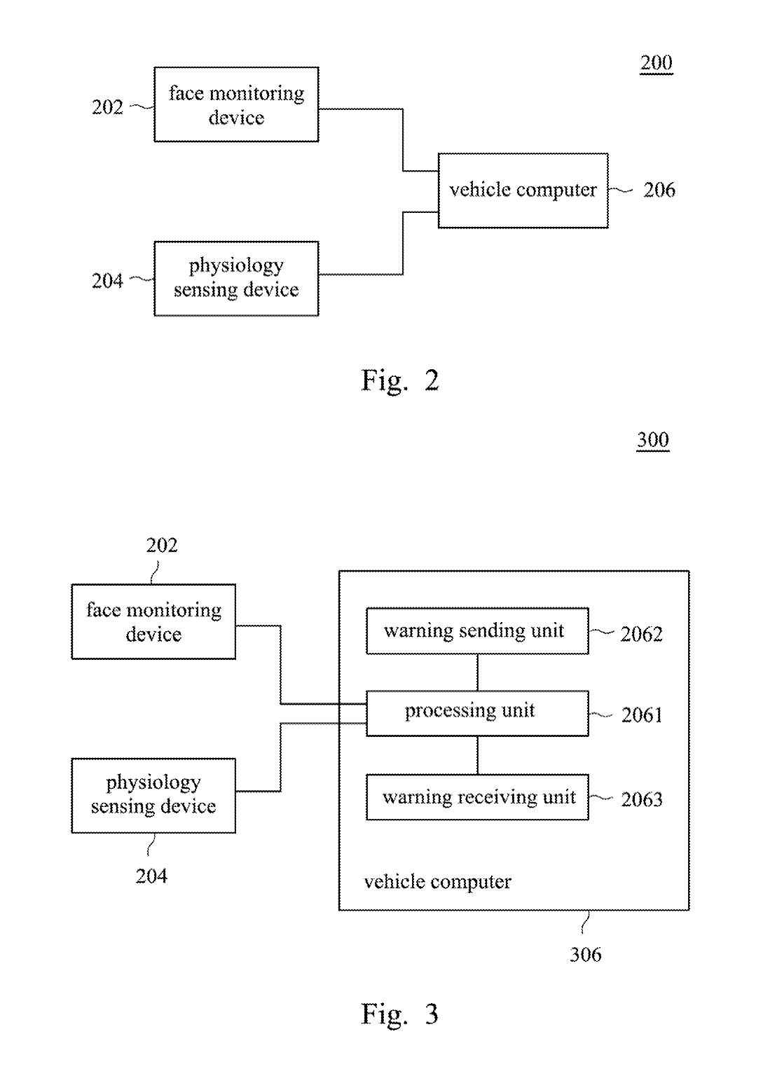

FIG. 2 is a schematic diagram illustrating a vehicle safety system 200 according to one embodiment of this disclosure. As illustrated in FIG. 2, the vehicle safety system 200 includes a face monitoring device 202, a physiology sensing device 204, and a vehicle computer 206. The vehicle computer 206 is electrically connected to the face monitoring device 202 and the physiology sensing device 204.

The step S102 is for monitoring a face state of a driver and determining whether the face state satisfies a tired state or not. The face monitoring device 202 may include a camera or other monitoring device. The face monitoring device 202 is configured to monitor the face state of the driver. The face state of the driver includes a mouse state of the driver, an open/close state of eyes of the driver, a head shaking state of the driver or a combination thereof. The face state is transmitted to the vehicle computer 206. The vehicle computer 206 analyzes the face state to determine whether the face state satisfies the tired state or not. For instance, if the eyes of the driver are almost closed, the driver may be sleeping or faint because he is tired. When the vehicle computer 206 determines the driver is tired, to proceed to step S104. When the vehicle computer 206 determines the driver is not tired, the face monitoring device 202 keeps monitoring.

The step S104 is for sensing a physiological state of the driver and determining whether the physiological state satisfies a conscious state or not. The physiology sensing device 204 is configured to sense the physiological state of the driver. The physiological state includes heartbeat, blood pressure or pulse etc. In some embodiments, the physiology sensing device 204 includes a wearable smart device, but is not limited thereof. The wearable smart device includes a smart bracelet, a smart watch, a smart clothes, a smart button, a smart necklace, etc. The physiological state is transmitted to the vehicle computer 206. The vehicle computer 206 may analyze the physiological state to determine whether the physiological state satisfies the conscious state or not. For instance, if the blood pressure of the driver is abnormally low, the driver may be unconscious because he is faint. When the vehicle computer 206 determines that the driver is tired and unconscious, the vehicle safety mechanism is directly activated to prevent accidents, as in step S112. On the contrary, when the vehicle computer 206 determines that the driver is tired but conscious (for example, the driver is sleeping), to proceed to step S106.

It is noted that the order of step S102 and 104 is not limited to FIG. 1. In other words, step S104 also can be ahead of step S102.

In step S106, the vehicle computer 206 sends a warning query if the face state of the driver satisfies the tired state and the physiological state of the driver satisfies the conscious state. Then, in step S108 and step S110, the vehicle computer 206 determines whether a response corresponding to the warning query is received or not. If the vehicle computer 206 doesn't receive the response (for example, the driver is sleeping), the vehicle computer 206 automatically activates the vehicle safety mechanism to protect the driver. In some embodiments, the vehicle safety mechanism includes an automatic brake mechanism, an automatic retardation mechanism, a lane correction mechanism or an alarm lamp lightening mechanism, etc. On the other hand, if the vehicle computer 206 receives the response (for example, the driver is tired but is awake), the face monitoring device 202 keeps monitoring, and the physiology sensing device 204 keeps sensing.

The above-mentioned vehicle safety method and vehicle safety system may send the warning query when the driver is tired and conscious. The vehicle safety mechanism may be configured to avoid the occurrence of an accident when the driver doesn't receive the warning query or doesn't response (for example, the driver is sleeping) the warning query. Moreover, the vehicle safety mechanism is directly activated to protect the driver when the driver is tired and unconscious.

FIG. 3 is a schematic diagram illustrating a vehicle safety system 300 according to another embodiment of this disclosure. As illustrated in FIG. 3, in some embodiments, a vehicle computer 306 is the vehicle computer 206. The vehicle computer 306 includes a processing unit 2061 a warning sending unit 2062, and a warning receiving unit 2063. The processing unit 2061 is electrically connected to the face monitoring device 202 and the physiology sensing device 204. The processing unit 2061 is further electrically connected to the warning sending unit 2062 and the warning receiving unit 2063. The processing unit 2061 may be a micro processor or other elements with processing and analyzing functions. The processing unit 2061 is configured to perform the determinations in step 5102 and in the step 5104 in FIG. 1. The warning sending unit 2062 is configured to send the warning query in step S106 in FIG. 1. In some embodiments, the warning query is sent out through a vocal method. At this time, the warning sending unit 2062 includes a loudspeaker. In further embodiments, the warning query may be a vocal file which is pre-recorded by the driver or by a family member of the driver. The vocal file may be stored in a storage unit of the vehicle computer 306 and be sent out by the warning sending unit 2062. Moreover, the warning receiving unit 2063 is configured to receive a response corresponding to the warning query. In some embodiments, the driver may send the response through the vocal method. At this time, the warning receiving unit 2063 may include a microphone. In some other embodiments, the driver sends the response through operating a button on a steering wheel. At this time, the warning receiving unit 2063 may include the button on the steering wheel, it is indicated that the driver is tired but conscious if the driver can send the response. On the other hand it is indicated that the driver is tired and sleeping if the driver can't send the response.

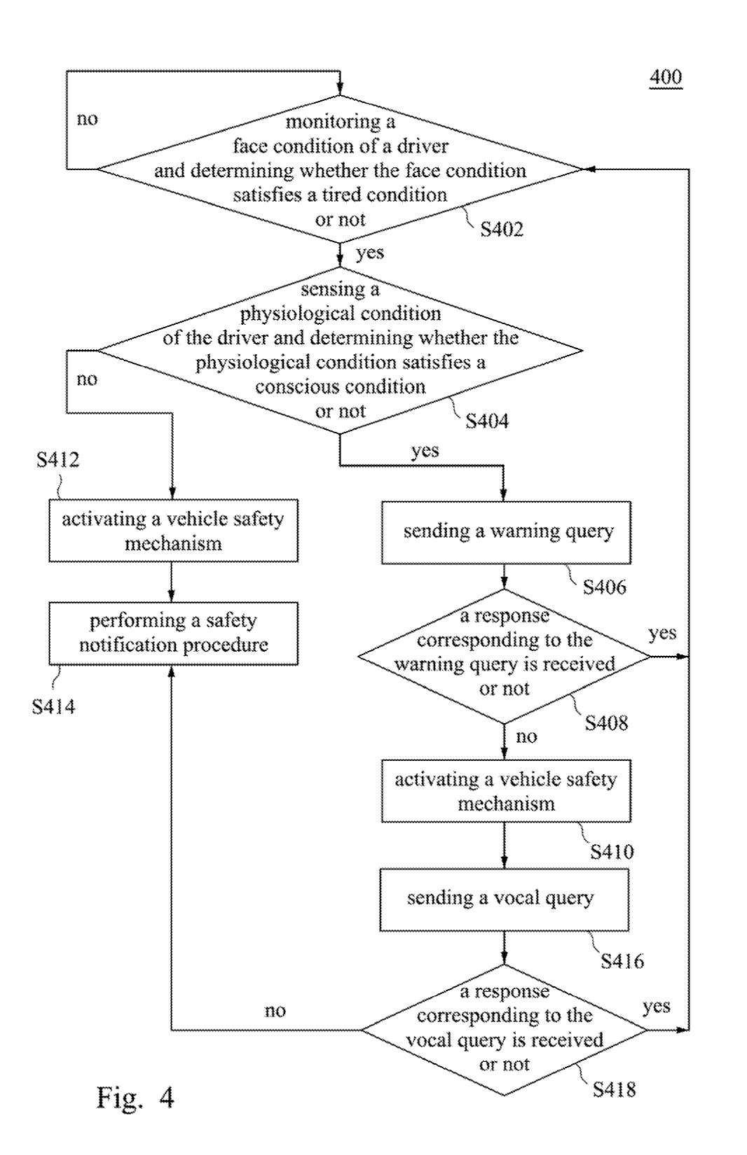

FIG. 4 is a flow diagram illustrating a vehicle safety method 400 according to yet another embodiment of this disclosure. The vehicle safety method 400 includes steps S402-S418. Steps S402-S412 are similar to the steps S102-S112 in FIG. 1, so a detailed description in this regard will not be provided.

The step S414 is for performing a safety notification procedure. In detail, when the face state of the driver satisfies the tired state and the physiological state doesn't satisfies the conscious state, not only the vehicle safety mechanism is activated, but also the safety notification procedure is performed. The safety notification procedure is performed to notify, for example, a police organization, a medical treatment organization or an emergent contact person. In some embodiments, a method of the performing the safety notification procedure may be implemented by a wireless network technology or a telephone communication technology.

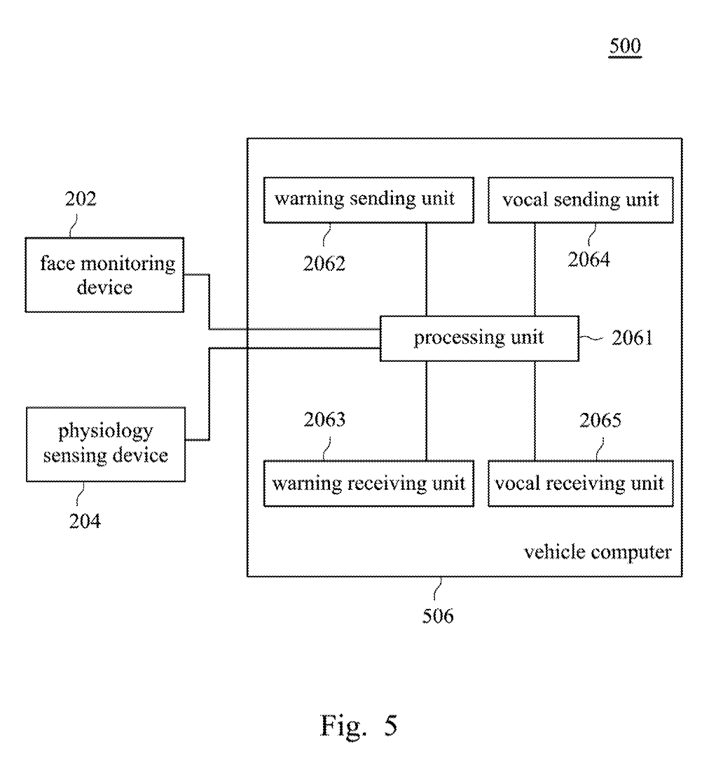

FIG. 5 is a schematic diagram illustrating a vehicle safety system 500 according to yet another embodiment of this disclosure. As illustrated in FIG. 5, the vehicle computer 506 is similar to the vehicle computer 306 in FIG. 3. In some embodiments, the vehicle computer 506 further includes a vocal sending unit 2064 and a vocal receiving unit 2065. The processing unit 2061 is electrically connected to the vocal sending unit 2064 and the vocal receiving unit 2065.

As illustrated in FIG. 4 and FIG. 5, in step S416, after the vehicle safety mechanism is activated, the vocal sending unit 2064 of the vehicle computer 506 may send out a vocal query. For instance, if the driver doesn't response the warning query in step S406, the vehicle safety mechanism in step S410 is activated, such as the car is stopped automatically. Then, the vocal sending unit 2064 may send the vocal query. The vocal query may be configured to awake the driver, and also may be configured to ask the driver whether the driver can continue to driver the car. If the vocal receiving unit 2065 receives the response from the driver, the processing unit 2061 may clear the vehicle safety mechanism. On the other hand, it is indicated that the driver doesn't be awaked due to other unknown reason if the vocal receiving unit 2065 doesn't receive the response from the driver. At this time, the vehicle computer 506 performs the safety notification procedure, as step S414, to notify, for example, a police organization, a medical treatment organization or an emergent contact person.

In some embodiments, the warning sending unit 2062 and the vocal sending unit 2064 may be implemented with the same element (such as, loudspeaker). In some embodiments, the warning receiving unit 2063 and the vocal receiving unit 2065 may be implemented with the same element (such as, microphone).

As the above embodiments, he vehicle safety method and the vehicle safety system send warning query when the driver is tired and conscious and activate the vehicle safety mechanism if the response corresponding to the warning query is not received. Consequently, the vehicle safety mechanism is configured to avoid an occurrence of an accident if the driver doesn't send the response (for example, the driver s sleeping).

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A vehicle safety method, comprising:
monitoring a face state of a driver and determining whether the face state satisfies a tired state or not;
sensing a physiological state of the driver and determining whether the physiological state satisfies a conscious state or not;
sending a warning query if the face state satisfies the tired state and the physiological state satisfies the conscious state;
determining whether a response corresponding to the warning query is received or not; and
activating a vehicle safety mechanism if the response corresponding to the warning query is not received.

2. The vehicle safety method of claim 1, further comprising:
activating the vehicle safety mechanism if the physiological state does not satisfy the conscious state.

3. The vehicle safety method of claim 1, wherein the response corresponding to the warning query is generated through inputting a voice by the driver or through operating a button on a steering wheel by the driver.

4. The vehicle safety method of claim 1, further comprising:
sending a vocal query after the vehicle safety mechanism is activated.

5. The vehicle safety method of claim 4, further comprising:
determining whether a response corresponding to the vocal query is received or not; and
performing a safety notification procedure if he response corresponding to the vocal query is not received.

6. The vehicle safety method of claim 1, wherein the vehicle safety mechanism comprises an automatic brake mechanism, an automatic retardation mechanism, a lane correction mechanism or an alarm lamp lightening mechanism.

7. The vehicle safety method of claim 1, wherein the physiological state is sensed through a wearable smart device.

8. A vehicle safety system, comprising:
a face monitoring device configured to monitor a face state of a driver;
a physiology sensing device electrically connected to the face monitoring device to sense a physiological state of the driver; and
a vehicle computer electrically connected to the face monitoring device and the physiology sensing device, wherein the vehicle computer is configured to determine whether the face state satisfies a tired state or not and determine whether the physiological state satisfies a conscious state or not, wherein the vehicle computer sends a warning query and determines whether a response corresponding to the warning query is received or not if the face state satisfies the tired state and the physiological state satisfies the conscious state, wherein the vehicle computer activates a vehicle safety mechanism if the response corresponding to the warning query is not received.

9. The vehicle safety system of claim 8, wherein the vehicle computer comprises:
a processing unit electrically connected to the face monitoring device to determine whether the face state satisfies the tired state or not, and electrically connected to the physiology sensing device to determine whether the physiological state satisfies the conscious state or not.

10. The vehicle safety system of claim 9, wherein the vehicle computer further comprises:
a warning sending unit electrically connected to the processing unit and configured to send the warning query.

11. The vehicle safety system of claim 10, wherein the vehicle computer further comprises:
a warning receiving unit electrically connected to the processing unit and configured to receive the response corresponding to the warning query.

12. The vehicle safety system of claim 9, wherein the vehicle computer further comprises:
a vocal sending unit electrically connected to the processing unit and configured to send a vocal query after the vehicle safety mechanism is activated.

13. The vehicle safety system of claim 12, wherein the vehicle computer further comprises:
a vocal receiving unit electrically connected to the processing unit and configured to receive a response corresponding to the vocal query.

14. The vehicle safety system of claim 8, wherein the physiology sensing device comprises a wearable smart device.

15. The vehicle safety system of claim 8, wherein the vehicle safety mechanism comprises an automatic brake mechanism, an automatic retardation mechanism, a lane correction mechanism or an alarm lamp lightening mechanism.
